(19) United States
(12) Patent Application Publication
(10) Pub. No.: US 2006/0233661 A1
(43) Pub. Date: Oct. 19, 2006

(54) Methods for disinfection and tracking of soiled items

(76) Inventor: Terrence R. Langford, Tucson, AZ (US)

Correspondence Address: QUARLES & BRADY STREICH LANG, LLP, ONE SOUTH CHURCH AVENUE, SUITE 1700, TUCSON, AZ, 85701-1621, US

(21) Appl. No.: 11/109,576

(22) Filed: Apr. 19, 2005

(51) Int. Cl. A61L 2/18

(52) U.S. Cl. 422/28

(57) ABSTRACT

Methods for single-step and rapid high-level or better disinfection utilizing a push/pull reprocessing apparatus and a predetermined mixture of detergent and sterilant. The accessible surfaces (including any lumens) of the soiled item or items to be disinfected are contacted with a mixture of detergent and sterilant or with detergent alone for a predetermined period followed by the addition of a sterilant. High-level disinfection or better is achieved within a 5 to 20 minute washing cycle period. A tracking function is also provided whereby the disinfection history of the item being reprocessed, the status of the reprocessing apparatus, whether the item is read-to-use, and the inventory of both items being reprocessed and the consumables used by the apparatus may be tracked.

### BACKGROUND OF THE INVENTION

1. Field of the Invention

This invention relates generally to improved methods involving push/pull reprocessing systems used to reprocess soiled items to an endpoint of high-level disinfection or better, and, more particularly, to methods that are especially useful in the reprocessing and tracking of information associated with soiled lumened medical items, such as endoscopes.

2. Description of the Related Art

The reprocessing (i.e., cleaning and decontamination) of items that come into contact with the bodily substances of people or animals such that they are substantially “substance free” (of, e.g., viruses, bacteria, detergent, sterilant, lipids, etc.) represent an immense and ongoing challenge. This challenge has been underscored by a recent article entitled “Widely used sterilizer under attack” (published in Jan. 21, 2003 edition of the newspaper USA Today). The article describes a fatal outbreak of bacterial infection that was linked to the improper sterilization of hospital bronchoscopes. Despite the hospital's use of one of the most popular sterilizing systems, tests performed by the Centers for Disease Control and Prevention found bacteria on the system's water filters and in its rinse water. This and other infection outbreaks has led to continuing controversy over how best to clean and sterilize used endoscopes.

The contaminants typically found on tubular or “lumened” medical items, such as endoscopes, are especially difficult to remove. In addition to fecal mater, loose cellular debris, blood and blood products, viruses, and bacteria, an endoscope can be coated with various hydrophobic films, such as “biofilm” material. A biofilm typically comprises cells, both dead and alive, cell debris and extracellular polymer substances. Once biofilm is formed by microorganisms (including bacteria, fungi, and protozoans), these microorganisms can colonize and replicate on the interior surfaces of tubing, forming a protective slime layer known as a “glycocalyx” that is especially difficult to remove.

Merely soaking endoscopes in a sterilant or detergent is unacceptable since numerous pockets exist within the tubing where the sterilant or detergent cannot reach effectively, which leaves areas of contamination within the endoscope. Moreover, with the prevalence of highly contagious diseases such as hepatitis B and C and Acquired Immune Deficiency Syndrome, reliable sterilization or disposal of all used medical tools seemingly becomes mandatory. Yet, while many medical instruments today are routinely cleaned, disinfected, and reused, experts in the field recently have warned that some of the more difficult to clean and sterilize medical items are putting people at risk.

Indeed, one expert has stated that there are no independent published reports or data anywhere in the medical literature that show liquid chemical sterilants (or any other method/process/agent) can be used to reliably “sterilize” flexible endoscopes or other complex, lumened instruments (See Comments by L. Muscarella (Custom Ultrasonics) on AAMI TIR7:1999, Chemical Sterilants and Sterilization Methods: A Guide to Selection and Use, downloaded from the website myendosite.com).

To the contrary, Kovacs et al. reports that a strain of Pseudomonas aeruginosa has been repeatedly isolated from tap water used for cleaning and rinsing endoscopes and appears to be responsible for three separate clinical episodes of endoscopic retrograde cholangio-pancreatography (ERCP)-associated cholangitis over an 11-yr period. These authors also conclude that the organism is resistant to a commonly used sterilant because it was recovered from a variety of endoscopes that had undergone stringent reprocessing protocols (see Kovacs B J, et al. “Efficacy of various disinfectants in killing a resistant strain of Pseudomonas aeruginosa by comparing zones of inhibition: Implications for endoscopic equipment reprocessing,” Am J Gastroenterol 1998; 93:2057-9). Thus, there is a genuine need for reliable methods of high-level disinfection or better to help ensure that even chemical-resistant pathogens are effectively eliminated.

Furthermore, some chemical cleaners or sterilants are so harshly reactive that they can damage the items they are meant to clean or sterilize. Thus, the problems encountered during item (and especially medical item) cleaning and disinfecting primarily involve trying to strike a balance between ensuring as much as possible the complete removal of contaminants and chemicals while, at the same time, not damaging the instrument.

After sterilization, endoscopes typically are rinsed with water filtered down to the 0.2 micron (200 nanometer) level. Unfortunately, many viruses, endotoxins, and prions are smaller than 200 nanometers, meaning that they can remain in the water even after filtration. Also, as reported in the articles mentioned above, water and water filters are known sources of contamination. Even more troubling, however, is the statement by one expert that “there are no independent data in the medical literature that support the production of sterile water (a biological endpoint defined as containing fewer than 10⁻⁶ CFU/ml or fewer than 5 endotoxin units/ml) by passing unprocessed water (that is, un-sterilized water, such as water that flows though a hospital's tap) through a bacterial (e.g., 0.1 or 0.2 micron) filtration system” (See Comments by L. Muscarella (Custom Ultrasonics) on AAMI TIR7:1999, Chemical Sterilants and Sterilization Methods: A Guide to Selection and Use, downloaded from the website myendosite.com). Moreover, there is no currently available system that monitors the biological content of filtered water to insure its sterility when used in conjunction with medical item cleaning or sterilization apparatuses. Finally, having to add additional sterilization steps and/or use sterilized (e.g., autoclaved) water becomes tedious and expensive.

So called “push/pull reprocessing systems” are automatic apparatuses that include a chamber containing a baffle with one or more openings through which water (or another fluid) surges back-and-forth (hence “push/pull”) through the opening or openings in the baffle. When soiled items, such as endoscopes and other lumened instruments, are placed within an opening, fluid also surges through them. Accordingly, a back-and-forth “scrubbing action” is created by the surging fluid the contacts any accessible surface on an item, including a lumen or lumens.

Such systems have been previously been described to provide superb cleaning based on physical inspection (for example, U.S. Pat. No. 5,753,195). However, as evidenced by the above media and scientific articles, methods for high-level or greater disinfection of soiled items have been a long sought after yet difficult to attain goal.

Therefore, there continues to be a need for a reprocessing method that, without damaging the item being treated, assists in loosening difficult soiling and provides for high-level disinfection or better of items in a relatively short amount of time.

### SUMMARY OF THE INVENTION

The invention generally involves single-step and short duration methods for high-level disinfection or better of soiled items placed within a push/pull reprocessing apparatus and contacted by a predetermined mixture of detergent and sterilant. The inventor has discovered, among other things, that using a mixture of detergent and sterilant decreases reprocessing time while reliably providing for high-level or better disinfection. The accessible surfaces of an item or items are contacted with a mixture of detergent and sterilant or with detergent alone for a predetermined period followed by the addition of a sterilant such that high-level disinfection or better is achieved within a 5 to 20 minute washing cycle period. A tracking function is also provided so that the status of the item being cleaned, the status of the reprocessing a apparatus, whether the item is ready-to-use, and the inventory of both the items being cleaned and consumables used by the reprocessing apparatus may be tracked.

An aspect of the invention is that the methods provide for a fast (5 to 20 minute) processing of soiled items such that they are reprocessed to a high level of disinfection or better by a mixture of one or more detergents and one or more sterilants being surged upon the accessible surfaces of the items in a push/pull reprocessing system. No other reprocessing system is known to disclose the invention's method steps and level of disinfection, particularly for lumened instruments.

An added point of novelty of this invention is an information or status tracking feature is preferably included to allow a user and others to track how many times a certain item has been reprocessed, whether a cleaning system has completed its cycle, whether an item is ready-to-use, and the quantity of consumables used or needed in a reprocessing system, among other things.

Accordingly, in one embodiment of the invention, a method for high-level disinfection or better of an item includes the steps of providing an item secured within a push/pull reprocessing system and contacting the item with a predetermined mixture of a detergent and a sterilant, wherein the item is cleaned to the level of 10⁻⁵ CFU/ml to 10⁻⁶ CFU/ml within 5 to 20 minutes, followed by the step of rinsing the item with ozonated sterile water.

In another embodiment of the invention, the method include the steps of providing a push/pull reprocessing system, placing an item within the reprocessing system, and contacting the item by surging a mixture of detergent and sterilant back-and-forth upon accessible surfaces of the item such that it is reprocessed to a biological endpoint of 10⁻⁵ CFU/ml to 10⁻⁶ CFU/ml within 5 to 20 minutes. In this and other embodiments, the mixture preferably includes about 50 vol/50 vol of detergent to sterilant (although other ratios of detergent to sterilant may be used efficaciously). Moreover, the contacting step may be performed first with detergent alone for a predetermined period, whereupon a sterilant is added to create a detergent/sterilant mixture before the cycle is complete. In this way, a detergent (e.g., an enzyme-based detergent) that may be degraded by a sterilant will have time to perform its function. Moreover, hybrid molecules containing both detergent and sterilant properties may utilized for the detergent and sterilant of the invention as such molecules become available in the future.

In another embodiment of the invention, a method for high-level disinfection or better is disclosed that includes the steps of contacting an item within a push/pull reprocessing system with a predetermined mixture of detergent and sterilant to a high-level disinfection or better endpoint of 10⁻⁵ CFU/ml to 10⁻⁶ CFU/ml in a single cycle (i.e., without any fluid draining or change-out). Preferably, the single cycle lasts between 5 to 20 minutes. An information tracking step also is preferably provided whereby a user and others can monitor information such as cleaning system status and item processing history in order to minimize the possibility that an item has been used and cleaned too many times or improperly cleaned (among other things). For example, a reprocessing technician may pre-rinse a endoscope to loosen soil and then leave it dry near endoscopes that have been cleaned and disinfected at the end of a shift. If the pre-rinsed endoscope appears clean by visual inspection, a technician on the subsequent shift may then mistakenly package it for re-use. The tracking step was specifically designed to prevent such mistakes by providing a means for indicating whether or not an endoscope (or other item) has been properly reprocessed before it can be used again.

Thus, it is a primary objective of the invention to provide a medical item reprocessing method that improves reliably ensures high-level disinfection or better while minimizing processing time, fluid change-out, and mistakes associated with item processing by tracking information associated with the reprocessing of an item.

In accordance with these and other objects, there is provided new and improved methods especially useful in the rapid high-level disinfection or better of lumened instruments.

Various other purposes and advantages of the invention will become clear from its description in the specification that follows. Therefore, to the accomplishment of the objectives described above, this invention includes the features hereinafter fully described in the detailed description of the preferred embodiments, and particularly pointed out in the claims. However, such description discloses only some of the various ways in which the invention may be practiced.

### DETAILED DESCRIPTION OF THE PREFERRED EMBODIMENTS

The invention relates generally to a methods for single-step and short duration high-level disinfection or better utilizing a push/pull reprocessing apparatus and a predetermined mixture of detergent and sterilant. The accessible surfaces of the item or items are contacted with a mixture of detergent and sterilant or with detergent alone for a predetermined period followed by a sterilant. High-level disinfection or better is achieved within a 5 to 20 minute washing cycle period. A tracking function is also provided whereby the status of the item being cleaned, the status of the cleaning a apparatus, the ready-to-use status of an item, and the inventory of both items being cleaned and consumables used by the cleaning apparatus may be tracked.

The terms “push/pull apparatus,” “push/pull system,” “cleaner/sterilizer,” “push/pull reprocessor,” “reprocessor,” and “reprocessing” as used throughout the specification are meant to be synonymous with the use of a push-pull apparatus (such as the Langford IC Systems, Inc. Manzi Mark 1) that cleans items (e.g., endoscopes, dental appliances, and the like) by surging fluid back-and-forth upon the accessible surfaces of these items.

Preferably, a step of rinsing the item or items being reprocessed with sterile ozonated water is provided. Because ozone is an unstable molecule, it should be generated at the point of application. It is generally formed by combining an oxygen atom with an oxygen molecule. This reaction is endothermic and requires a considerable input of energy. Ozone can be produced several ways, although one method, corona discharge, predominates in the ozone generation industry. Ozone can also be produced by irradiating an oxygen-containing gas with ultraviolet light, electrolytic reaction and other emerging technologies. Most ozone generators currently use ultraviolet radiation. These are usually the lowest cost ozone generators on a per unit basis. This decrease in cost is due to the fact that the air does not go through an initial drying process.

Newer units being produced utilize a corona discharge technique which dry the air before charging the air with ozone. This drying permits the corona discharge apparatus to produce a higher ozone concentration. For minimal expenditures of electrical energy, ozone normally is produced from dried air (−60 degrees Fahrenheit dew point) in concentrations of one to two percent and from dry oxygen in concentrations of two to four percent. More than eighty percent of the electrical energy applied to the electric discharge field is converted to heat and, if this is not quickly removed from the cell, the heat causes rapid decomposition of the ozone back to oxygen. For additional guidance in ozone production and its uses, see U.S. Pat. No. 5,207,237.

For methods of the invention involving the use of an ozonated water rinse, it is important to recognize that the time of exposure and concentration of ozone will vary based on a number of parameters, such as the quantity and size of items being treated, and the volume of the cleaning or sterilizing apparatus. Preferably, ozonated water is used to rinse items for 1-10 minutes at a concentration of 1-10% ozone by volume.

A number of known cleaning and sterilization methods are readily available. Some are performed in automatic endoscope reprocessors, while others are done manually. For example, Yale Medical School recommends that an endoscope can be manually cleaned by placing the distal end of the endoscope into an enzymatic detergent solution followed by applying suction to the solution through the biopsy/suction channel until the solution is visibly clean. One then alternates the suctioning of clean detergent solution with air several times, followed by removing the air under vacuum (further details are available online at Yale's Internet website info.med.yale.edu/ynhh/infection/steril/standards).

However, whether high-level disinfection or better is reliably achieved and the amount of time and washing cycles (and/or fluid change-outs) make such methods burdensome and questionable in the face of the media and scientific literature described above.

Within the following examples, endoscopes will be used as an example of an item or instrument to be reprocessed. However, the inventor contemplates use of the invention with any tubular item, as well as a variety of other items such as circuit boards, medical instruments, dental instruments, and other items in which reliable cleaning and high-level disinfection or better is required.

### EXAMPLE 1

The purpose of this test is to document the results of engineering characterization testing performed on a automatic endoscope reprocessor, the Langford I.C. Systems Sterilizer Cleaner (see U.S. Pat. No. 5,906,802 for layout and guidance in the use of this reprocessor).

Therefore, this test seeks to describe methods and test results for reprocessing efficacy of individual phases on mock devices used to simulate a colonoscope. Testing was performed on DWGX-0129-01888, Cleaner, Sterilizer Breadboard.

The biopsy lumen of three colonoscopes were loaded with Hucker's Soil (much more than required by FDA test standards) and inoculated with pathogens from an American Society of Test Methods kit. The scopes were left sitting for a 24 hour time period to permit some drying. Using the same Langford I.C. Systems Sterilizer Cleaner liquid-displacement settings as described, each colonoscope was subjected to one wash cycle at 4 psi for 5 min with a 50%/50% (vol/vol) mixture of SIMPLE GREEN cleaner and peracetic acid sterilant in 10 liters of water.

Tests performed to quantify the level of decontamination on the three mock scopes used in this example indicated that two of the scopes showed a log 10⁻⁵ pathogen kill while one scope had log 10⁻⁶ pathogen kill (indicates sterility). Visual inspection revealed no apparent damage to any endoscope surface.

Especially given the extremely high level of soiling, these results are much better than has previously been achieved for any known cleaning/disinfecting protocol based on a 10 minute cycle.

The Langford I.C. Systems Sterilizer Cleaner performed effectively at pressures as little as 4 psi and at temperatures as low as 50° F. for cycles of as little as 5 minutes in length. Indeed, the ability to use “cool” to “cold” water between 100° F. and 50° F. saves on the cost over using warmer water. The preferred rate of “liquid displacement” (i.e., the back-and-forth liquid cycling rate in the item-washing chamber of the Sterilizer Cleaner) is 1 gallon per 2 seconds.

After the detergent and sterilant cycle is complete, preferably water is ozonated by corona discharge and applied to the item. Thus, for example, the mock scopes may be rinsed with the ozonated water for 5 minutes to degrade any residual detergent followed by three 5 min rinses with 10 liters of filtered tap water. For the last (third) rinse, ozone generated by corona discharge was added to the water thru a Mazzei venturi injector connected to the incoming water line of the Langford reprocessor apparatus. The ozone is added to the water at a rate of 1.25 g/hr at 5 SCFH dry air flow.

### EXAMPLE 2

In this example, a cleaning/sterilizing device of the type described and illustrated in U.S. Pat. No. 5,711,921 is utilized. The endoscope is positioned to extend through the baffle of the device such that one opening of the endoscope lies in one chamber and another opening of the endoscope lies in the other chamber. The baffle between the chambers need not be an absolute partition and, in this example, the baffle fits loosely around the endoscope so that as the fluid medium (e.g., a liquid detergent, sterile water, a liquid sterilant) surges from one chamber to the other, the medium washes over the exterior of the endoscope and simultaneously sweeps through the interior of the endoscope. The device creates this “surge” through the use of one or more flexible membranes. By deforming the flexible membrane (inward and outward), a pressure or suction is created which results in a flow (liquid displacement) between the chambers to equalize the pressure between them.

Next, 250 ml of detergent is added to 10 liters of water and is used to wash the endoscope for 10 min. After 5 minutes of the first cycle, 10 liters of a liquid chemical sterilant (preferably 1 ounce paracetic acid per 5 liters of water) are added to the cleaner/sterilizer and the endoscope continues to be treated for 5 minutes. Those of ordinary skill in the art readily recognize various other sterilants which can be used in this context.

The invention also pertains to the tracking of information associated with an item or items being reprocessed. For example, in order to know the structural integrity information of an item such as a endoscope, there is a need to track its reprocessing history. This is because such scopes are only reusable a certain number of times before the risk of structural compromise becomes significant.

To accomplish this information tracking, different methods of coding and reading exist in order to identify the history of a particular item (e.g., a endoscope) during a particular time in the reprocessing operation. Such methods of tracking may include, for example, utilizing bar coding and scanner technology and radio-frequency tagging such that the identity and cleaning history of any scope is determined by an acquisition device (i.e., a “reader”) in or around the push/pull reprocessing device.

Prophetic Examples

1. A colonoscope used for a procedure contains a radio frequency tag that is read by the reprocessor device via radiowaves over a Bluetooth network when the scope is brought into proximity with the reprocessor. In this instance, a computer processor associated with the network determines that this particular colonoscope has already been reprocessed five times previously. Therefore, a message warning that reprocessing is not recommended due to reaching of the manufacturer's recommended limit on use and the colonoscope is thrown away.

2. Thirty endoscopes have been run through a computer-automated push/pull apparatus in a given period. Each time an endoscope is removed, the apparatus operator runs a bar code embedded in each scope by an optical scanner. Because the optical scanner is linked with a computer processor that tracks inventory for the consumables (e.g., detergent and sterilant) used during each reprocessing operation, the computer of the apparatus alerts the operator that such consumables must be replaced before a new reprocessing operation begins to assure that a given item is exposed to a required or recommended level of consumables.

3. A bronchoscope used for a procedure contains a radio frequency tag that is read by the detector when the scope is brought into proximity with a treatment area. In this instance, a computer processor associated with the detector network determines that, although this particular bronchoscope appears to be clean, it has not been run through a reprocessing system since its last use. Therefore, a message warning that the bronchoscope is not safe to use is conveyed to the medical practitioner.

Various changes in the details and components that have been described may be made by those skilled in the art within the principles and scope of the invention herein described in the specification and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products. All references cited in this application are hereby incorporated by reference herein.

I claim:

1. A method for achieving at least high-level disinfection of a soiled item, comprising the steps of:
a. securing said soiled item within a push/pull reprocessing system;
b. flowing a predetermined mixture including a fluid, a detergent, and a sterilant over accessible surfaces of the soiled item, wherein the item is reprocessed to a biological endpoint of 10⁻⁵ CFU/ml to 10⁻⁶ CFU/ml; and
c. flowing ozonated sterile water over said accessible surfaces after step (b).

2. The method of claim 1, wherein said biological endpoint of 10⁻⁵ CFU/ml to 10⁻⁶ CFU/ml is achieved within 5 to 20 minutes.

3. The method of claim 1, wherein the fluid of step (b) is between 50° F. to 100° F.

4. The method of claim 1, wherein step (b) is performed for a duration and with a mixture of detergent and sterilant such that less than 6.4 ug/cm² of a test protein remains upon said item.

5. The method of claim 4, wherein said mixture comprises about 50vol/50vol of detergent to sterilant and said duration is between 5 and 10 minutes.

6. The method of claim 1, wherein step (b) is performed with said detergent alone for a predetermined period, and said sterilant is subsequently added to said mixture before step (b) has ended.

7. The method of claim 1, further including a step of tracking information associated with said soiled item.

8. The method of claim 7, wherein said information is selected from the group consisting of one or more of: a number of times said item has been processed for cleaning, confirmation that a process for cleaning has been completed, whether said item is ready-to-use, and inventory tracking of said item, said detergent, or said sterilant used in cleaning said item.

9. The method of claim 1, wherein said soiled item comprises a lumened instrument.

10. A method for high-level disinfection or better of a soiled item, comprising the steps of:
a. providing a push/pull reprocessing system;
b. securing said item within said reprocessing system;
c. flowing a mixture including a fluid, a detergent, and a sterilant back-and-forth upon accessible surfaces of said soiled item for at least 5 minutes, wherein the item is reprocessed to a biological endpoint of 10⁻⁵ CFU/ml to 10⁻⁶ CFU/ml; and
d. flowing ozonated sterile water over said accessible surfaces after step (c).

11. The method of claim 10, wherein said biological endpoint of 10⁻⁵ CFU/ml to 10⁻⁶ CFU/ml is achieved within 5 to 20 minutes.

12. The method of claim 10, wherein the fluid of step (c) is between 50° F. to 100° F.

13. The method of claim 10, wherein step (c) is performed for a duration and with a mixture of detergent and sterilant such that less than 6.4 ug/cm² of a test protein remains upon said item.

14. The method of claim 13, wherein said mixture comprises about 50vol/50vol of detergent to sterilant and said duration is between 5 and 10 minutes.

15. The method of claim 10, wherein step (c) is performed with said detergent alone for a predetermined period, and said sterilant is subsequently added to said mixture before step (b) has ended.

16. The method of claim 10, further including a step of tracking information associated with said soiled item.

17. The method of claim 16, wherein said information is selected from the group consisting of one or more of: a number of times said item has been processed for cleaning, confirmation that a process for cleaning has been completed, whether said item is ready-to-use, and inventory tracking of said item, said detergent, or said sterilant used in cleaning said item.

18. The method of claim 10, wherein said item comprises a lumened instrument.

19. A method for high-level disinfection or better of a soiled item secured within a push/pull reprocessing apparatus, comprising the step of:
a. contacting accessible surfaces of said soiled item with a predetermined mixture including a fluid, a detergent, and a sterilant to a biological endpoint of 10⁻⁵ CFU/ml to 10⁻⁶ CFU/ml in a single washing cycle of 20 minutes or less.

20. The method of claim 19, further including the step of:
b. flowing ozonated sterile water over said accessible surfaces after step (a).

21. The method of claim 19, wherein the fluid of step (a) is between 50° F. to 100° F.

22. The method of claim 19, wherein step (a) is performed for a duration and with a mixture of detergent and sterilant such that less than 6.4 ug/cm² of a test protein remains upon said item.

23. The method of claim 22, wherein said mixture comprises about 50vol/50vol of detergent to sterilant and said duration is between 5 and 10 minutes.

24. The method of claim 19, wherein step (a) is performed with said detergent alone for a predetermined period, and said sterilant is subsequently added to said mixture before step (a) has ended.

25. The method of claim 19, further including a step of tracking information associated with said soiled item.

26. The method of claim 19, wherein said information is selected from the group consisting of one or more of: a number of times said item has been processed for cleaning, confirmation that a process for cleaning has been completed, whether said item is ready-to-use, and inventory tracking of said item, said detergent, or said sterilant used in cleaning said item.

27. The method of claim 19, wherein said item comprises a lumened instrument.
